(19) United States
(12) Patent Application Publication
(10) Pub. No.: US 2022/0054003 A1
(43) Pub. Date: Feb. 24, 2022

(54) POLARIZED VISUAL ACUITY CHART PROJECTOR

(71) Applicant: HUVITZ CO., LTD., Anyang-si (KR)

(72) Inventor: Min Soo Cho, Anyang-si (KR)

(21) Appl. No.: 17/392,362

(22) Filed: Aug. 3, 2021

(30) Foreign Application Priority Data
Aug. 20, 2020 (KR) 10-2020-0104771

(51) Int. Cl. A61B 3/032; A61B 3/08

(52) U.S. Cl. CPC A61B 3/032; A61B 3/08

(57) ABSTRACT

Disclosed is a polarized visual acuity chart projector for generating visual acuity charts polarized differently for a binocular vision examination. The polarized visual acuity chart projector comprises: a first image display device configured to project a first visual acuity chart polarized in a first direction; a second image display device configured to project a second visual acuity chart polarized in a second direction; and an optical splitter configured to transmit the first visual acuity chart emitted from the first image display device as it is to thereby project the first visual acuity chart polarized in the first direction in the direction of an examinee, and configured to reflect the second visual acuity chart polarized in the second direction emitted from the second image display device to thereby project a second visual acuity chart polarized in a third direction different from the first direction in the direction of the examinee.

### CROSS-REFERENCE TO RELATED APPLICATION

This application claims benefit of priority to Korean Patent Application No. 10-2020-0104771 filed on Aug. 20, 2020, the entire contents of which are incorporated herein by reference.

### TECHNICAL FIELD

The present invention relates to a polarized visual acuity chart projector, and more particularly, to a polarized visual acuity chart projector for generating visual acuity charts polarized differently for a binocular vision examination.

### BACKGROUND

Binocular vision is a term that contrasts monocular vision. Binocular vision refers to a method of recognizing an object perceived respectively by both eyes as a single object by the cooperative action of both eyes when gazing at the same location of the object with both eyes. Since the positions of the left and right eyes differ by about 6 cm, the images reflected in the left and right eyes are slightly different even when looking at the same object, which is called binocular parallax (disparity). In spite of this parallax, what we see in general is a single fused image rather than images such as those superimposed, which is called binocular fusion. In other words, if objects of similar shapes are presented to the right and left eyes, respectively, the brain recognizes the existence of one object by fusing the images perceived by both eyes. On the other hand, if objects of completely different shapes are presented to both eyes, the images perceived by both eyes will be recognized separately instead of being fused into one. This is called binocular rivalry.

In order to examine such a binocular vision function, polarized visual acuity chart projectors in which visual acuity charts are intended to be projected to the right eye and the left eye of an examinee, respectively, are known. FIG. 1 is a diagram for illustrating the operating principle of a typical polarized visual acuity chart projector for a binocular vison examination. As shown in FIG. 1, the conventional polarized visual acuity chart projector comprises an image display device 10 such as an LCD that displays a visual acuity chart of a predetermined shape, and a polarizing film 20 of a sheet shape attached to the front surface of the image display device 10. Polarization regions 20a and 20b of a line shape extending in one direction (horizontal direction in FIG. 1) in correspondence with the pixel size of the image display device 10 are alternately arranged in the polarizing film 20. The polarization regions 20a and 20b comprise a first polarization region 20a and a second polarization region 20b to generate a pair of chart images having polarization axes orthogonal to each other when transmitting the light of a chart image emitted from the image display device 10. The first polarization region 20a is an optical region for the right eye, and the direction of its optical principal-axis is arranged so as to be converted into polarized light having a polarization axis direction (135-degree direction) that coincides with the polarization axis direction of a polarization filter 22a for the right eye. On the other hand, the second polarization region 20b is an optical region for the left eye, and the direction of its optical principal-axis is arranged so as to be converted into polarized light having a polarization axis direction (45-degree direction) that coincides with the polarization axis direction of a polarization filter 22b for the left eye.

In such a polarized visual acuity chart projector, when the image display device 10 is operated so that desired chart shapes are displayed on the first polarization region 20a and the second polarization region 20b, and the polarization filter 22a for the right eye and the polarization filter 22b for the left eye are mounted to the right eye and the left eye of the examinee, only the chart images of the first polarization region 20a and the second polarization region 20b are shown to the right and left eyes of the examinee, respectively, and thus, a binocular vision examination can be performed by examining how the examinee recognizes them.

However, the polarizing film 20 for a linearly polarized LCD of a line unit described above has problems of being expensive and difficult to manufacture, as well as likely to cause process defects when attaching the manufactured polarizing film 20 to the image display device 10.

### Prior Art Literature

Korean Patent Publication No. 10-2011-0035876 (Apr. 6, 2011)

### SUMMARY

### Technical Objects

Therefore, it is an object of the present invention to provide a polarized visual acuity chart projector that does not use a polarizing film.

It is another object of the present invention to provide a polarized visual acuity chart projector that can be manufactured with commercially easily available parts, has a simple manufacturing process, and can reduce manufacturing costs.

### Technical Solution

In order to achieve the objects above, the present invention provides a polarized visual acuity chart projector comprising: a first image display device 30a configured to project a first visual acuity chart 40a polarized in a first direction; a second image display device 30b configured to project a second visual acuity chart 40b polarized in a second direction; and an optical splitter 50 configured to transmit the first visual acuity chart 40a emitted from the first image display device 30a as it is to thereby project the first visual acuity chart 40a polarized in the first direction in the direction of an examinee, and configured to reflect the second visual acuity chart 40b polarized in the second direction emitted from the second image display device 30b to thereby project a second visual acuity chart 40b polarized in a third direction different from the first direction in the direction of the examinee.

### Effects of the Invention

The polarized visual acuity chart projector in accordance with the present invention does not use a polarizing film, can be manufactured with commercially easily available parts, has a simple manufacturing process, and can reduce manufacturing costs.

### BRIEF DESCRIPTION OF THE DRAWINGS

FIG. 1 is a diagram for illustrating the operating principle of a typical polarized visual acuity chart projector for a binocular vison examination;

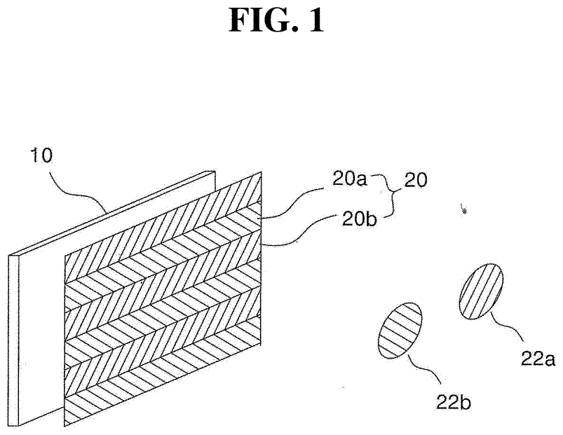

FIG. 2 is a diagram showing the structure of a polarized visual acuity chart projector in accordance with one embodiment of the present invention; and

FIG. 3 is a diagram showing a state in which an examinee recognizes visual acuity charts emitted from a polarized visual acuity chart projector in accordance with one embodiment of the present invention.

### DETAILED DESCRIPTION

Hereinafter, the present invention will be described in detail with reference to the accompanying drawings. In the drawings attached, the same reference numerals are assigned to elements that perform the same or similar functions as in the prior art.

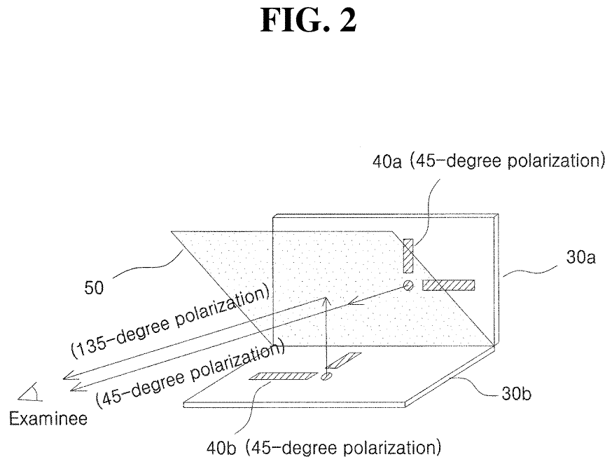

FIG. 2 is a diagram showing the structure of a polarized visual acuity chart projector in accordance with one embodiment of the present invention. As shown in FIG. 2, the polarized visual acuity chart projector in accordance with the present invention includes a first image display device 30a, a second image display device 30b, and an optical splitter 50.

The first image display device 30a projects a first visual acuity chart 40a polarized in a first direction, and the second image display device 30b projects a second visual acuity chart 40b polarized in a second direction. The optical splitter 50 transmits the first visual acuity chart 40a emitted from the first image display device 30a as it is to thereby project the first visual acuity chart 40a polarized in the first direction in the direction of an examinee, and reflects the second visual acuity chart 40b polarized in the second direction emitted from the second image display device 30b to thereby project a second visual acuity chart 40b polarized in a third direction different from the first direction in the direction of the examinee.

For a binocular vision examination, since the first visual acuity chart 40a that is emitted from the first image display device 30a and is projected in the direction of the examinee should be recognized by only one of both eyes of the examinee and the second visual acuity chart 40b that is emitted from the second image display device 30b and is projected in the direction of the examinee should be recognized by only the other of both eyes of the examinee, the first polarization direction of the first visual acuity chart 40a and the third polarization direction of the second visual acuity chart 40b should be different from each other. For example, as shown in FIG. 2, if the first polarization direction of the first visual acuity chart 40a emitted from the first image display device 30a is 45 degrees, then the polarization direction of the first visual acuity chart 40a that has passed through the optical splitter 50 is also 45 degrees, and thus, the examinee recognizes the first visual acuity chart 40a polarized at 45 degrees. On the other hand, if the second polarization direction of the second visual acuity chart 40b emitted from the second image display device 30b is 45 degrees, then the polarization direction (third direction) of the second visual acuity chart 40b that has been specular-reflected by the optical splitter 50 is 135 degrees, and thus, the examinee recognizes the second visual acuity chart 40b polarized at 135 degrees. If polarized light is reflected by a mirror, its polarization angle is changed. For example, if light polarized at 45 degrees is reflected by a mirror at a reflection angle of 90 degrees, then light polarized at 135 degrees is reflected. The present invention generates the charts 40a and 40b with different polarization directions using the characteristic that, for example, if polarized light is reflected by a 45-degree reflection mirror, the polarization angle is symmetrically inverted by 90 degrees.

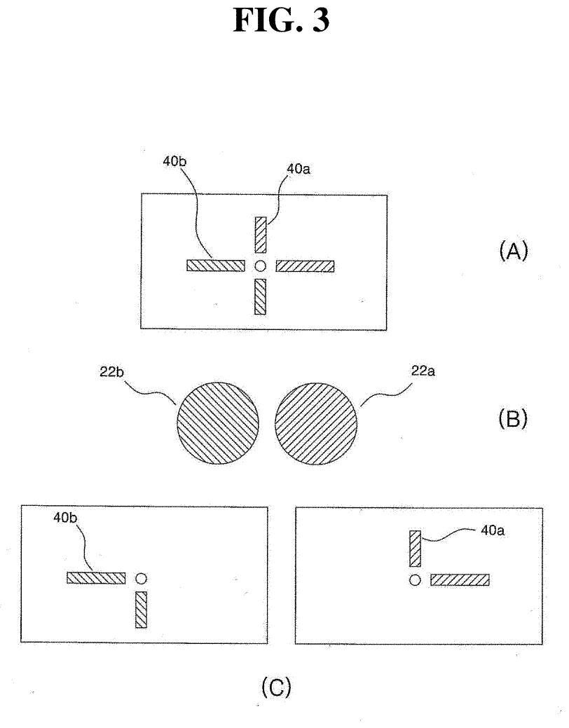

FIG. 3 is a diagram showing a state in which an examinee recognizes visual acuity charts emitted from a polarized visual acuity chart projector in accordance with one embodiment of the present invention. As shown in A of FIG. 3, a polarized visual acuity chart projector in accordance with the present invention projects a first visual acuity chart 40a polarized in a first polarization direction (45 degrees in FIG. 3) and a second visual acuity chart 40b polarized in a third polarization direction (135 degrees in FIG. 3). At this time, if a polarization filter 22a for the right eye polarized in the first polarization direction (45 degrees in FIG. 3) is mounted to the right eye of an examinee (B of FIG. 3), then the first visual acuity chart 40a polarized in the first polarization direction (45 degrees in FIG. 3) is recognized by the right eye of the examinee, but the second visual acuity chart 40b polarized in the third polarization direction (135 degrees in FIG. 3) is blocked and cannot be recognized (C of FIG. 3). Likewise, if a polarization filter 22b for the left eye polarized in the third polarization direction (135 degrees in FIG. 3) is mounted to the left eye of the examinee (B of FIG. 3), then the second visual acuity chart 40b polarized in the third polarization direction (135 degrees in FIG. 3) is recognized by the left eye of the examinee, but the first visual acuity chart 40a polarized in the first polarization direction (45 degrees in FIG. 3) is blocked and cannot be recognized (C of FIG. 3). Therefore, by projecting different charts to the right eye and the left eye of the examinee, the binocular vision examination can be performed for the examinee.

In the polarized visual acuity chart projector in accordance with the present invention, the difference between the first polarization direction of the first visual acuity chart 40a and the third polarization direction of the second visual acuity chart 40b is sufficient enough if one of the first visual acuity chart 40a and the second visual acuity chart 40b can be recognized only by the right eye and the other can be recognized only by the left eye by mounting the polarization filter 22a for the right eye and the polarization filter 22b for the left eye with different polarization directions to the right eye and left eye of the examinee. If the first polarization direction and the third polarization direction are the same or the difference therebetween is small (for example, 45 degrees or less), images of charts other than the intended charts can be projected onto each of the eyes to be examined even if the polarization filter 22a for the right eye and the polarization filter 22b for the left eye with different polarization directions are used. Preferably, it is desirable when there is a 90-degree difference between the first polarization direction and the third polarization direction and there is also a 90-degree difference between the polarization directions of the polarization filter 22a for the right eye and the polarization filter 22b for the left eye because images of charts other than the intended charts can be most completely blocked from being projected.

The first image display device 30a and the second image display device 30b may be any image display device as long as they can project the first visual acuity chart 40a and the second visual acuity chart 40b that are polarized. If the image display device is an image display device that does not have polarization such as OLEDs, a polarization filter polarized at a predetermined angle may be attached to the image display device, so as to be used as the first image display device 30a and the second image display device 30b. If the image display device is a liquid crystal display device (LCD) or the like that projects a polarized image by itself by including a polarizing film in its internal structure, it can be used as it is without attaching a separate polarizing film. For example, since small (5.7/5 inch) LCDs used in smartphones, etc. generally have a polarization angle of 45 degrees, it can be used as it is without attaching a separate polarizing film. However, if the degree of self-polarization shielding of a commercially available LCD module is not high, a polarizing film of a corresponding angle may also be additionally attached and used.

The optical splitter 50 is a typical device that reflects some of incident light and transmits some of it, may be, for example, a pair of prisms joined in a hexahedral shape, half mirrors, dichroic mirrored prisms, etc., and transmits 50% of incident light and reflects 50% of it, for example.

In a preferred embodiment of the present invention, the first image display device 30a and the second image display device 30b are arranged so as to face each other at an angle of 90 degrees, and the optical splitter 50 is arranged between the first image display device 30a and the second image display device 30b at an angle of 45 degrees, as shown in FIG. 2. Further, the first image display device 30a and the second image display device 30b may be any image display device as long as they can project the first visual acuity chart 40a and the second visual acuity chart 40b that are polarized at 45 degrees. In this case, the first visual acuity chart 40a polarized at 45 degrees passes through the optical splitter 50 and thus, is projected onto the examinee as it is, and the second visual acuity chart 40b polarized at 45 degrees is reflected by the optical splitter 50 and thus, the second visual acuity chart 40b polarized at 135 degrees is projected onto the examinee. In this case, it is desirable because the first visual acuity chart 40a polarized at 45 degrees and the second visual acuity chart 40b polarized at 135 degrees are delivered to the examinee in parallel to each other.

Although the present invention has been described above with reference to exemplary embodiments, the present invention is not limited to the embodiments described above. The scope of the claims that follow should be construed to encompass all variations, equivalent constructions and functions of the exemplary embodiments.

What is claimed is:

1. A polarized visual acuity chart projector comprising:
a first image display device (30a) configured to project a first visual acuity chart (40a) polarized in a first direction;
a second image display device (30b) configured to project a second visual acuity chart (40b) polarized in a second direction; and
an optical splitter (50) configured to transmit the first visual acuity chart (40a) emitted from the first image display device (30a) as it is to thereby project the first visual acuity chart (40a) polarized in the first direction in the direction of an examinee, and configured to reflect the second visual acuity chart (40b) polarized in the second direction emitted from the second image display device (30b) to thereby project a second visual acuity chart (40b) polarized in a third direction different from the first direction in the direction of the examinee.

2. The polarized visual acuity chart projector of claim 1, wherein the first image display device (30a) and the second image display device (30b) are liquid crystal display devices that project polarized images by themselves.

3. The polarized visual acuity chart projector of claim 1, wherein the first image display device (30a) and the second image display device (30b) are image display devices capable of projecting the first visual acuity chart (40a) and the second visual acuity chart (40b) that are polarized at 45 degrees, and the second visual acuity chart (40b) polarized at 45 degrees is reflected by the optical splitter (50) and thus, the second visual acuity chart (40b) polarized at 135 degrees is projected onto the examinee.

4. The polarized visual acuity chart projector of claim 1, wherein a polarization filter (22a) for a right eye polarized in the first direction is mounted to the right eye of the examinee, so that the first visual acuity chart (40a) polarized in the first direction is recognized by the right eye of the examinee and the second visual acuity chart (40b) polarized in the third direction is blocked and cannot be recognized, and a polarization filter (22b) for a left eye polarized in the third direction is mounted to the left eye of the examinee, so that the second visual acuity chart (40b) polarized in the third direction is recognized by the left eye of the examinee but the first visual acuity chart (40a) polarized in the first direction is blocked and cannot be recognized.

5. The polarized visual acuity chart projector of claim 1, wherein the difference between a first polarization direction of the first visual acuity chart (40a) and a third polarization direction of the second visual acuity chart (40b) is set such that one of the first visual acuity chart (40a) and the second visual acuity chart (40b) is recognized only by a right eye and the other is recognized only by a left eye by mounting a polarization filter (22a) for the right eye and a polarization filter (22b) for the left eye having different polarization directions to the right eye and left eye of the examinee.
